(19) United States
(12) Patent Application Publication
(10) Pub. No.: US 2023/0118660 A1
(43) Pub. Date: Apr. 20, 2023

(54) USE OF PHYTASE TO OBTAIN IMPROVED FOOD

(71) Applicant: DSM IP ASSETS B.V., Heerlen (NL)

(72) Inventors: Monica Diana VLASIE, Echt (NL); Pieter Cornelis LANGEVELD, Echt (NL)

(21) Appl. No.: 17/906,118

(22) PCT Filed: Mar. 29, 2021
(86) PCT No.: PCT/EP2021/058137

§ 371 Date: Sep. 12, 2022

(30) Foreign Application Priority Data
Mar. 31, 2020 (EP) 20167149.2

(51) Int. Cl. A23J 3/14; A23L 11/65; A23L 11/60; A23L 2/38; A23L 2/66; A23L 11/30; A23C 11/10; A23L 7/104; A23L 25/00; A23L 33/185; A23J 3/16; A23L 29/00; A23C 20/02; C12N 9/16

(52) U.S. Cl. CPC A23J 3/14; A23L 11/65; A23L 11/60; A23L 2/382; A23L 2/66; A23L 11/33; A23C 11/103; A23C 11/10; A23L 7/107; A23L 25/40; A23L 33/185; A23J 3/16; A23L 29/06; A23C 20/025; C12N 9/16

(57) ABSTRACT

The present invention relates to the food field. The invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein.

### FIELD

The present invention relates to the food field.

### BACKGROUND

In a world with a growing population there is an increased demand of proteins. To respond to this growing demand of proteins, there is a need to look at wider applications of proteins. In addition, it is desired to look for plant proteins as an alternative for animal proteins, since it is considered that plants are a more sustainable source of proteins than animals. The use of plant proteins in food is still limited in part because of poor texture and taste.

Foods comprising plant-based protein often are astringent and have quite a pronounced off taste. This limits the expansion of plant-based protein foods in the market.

In addition to obtain a good structure and prevent synereses of a plant-based beverages (fermented and non-fermented), stabilizers like hydrocolloids are needed with unwanted labelling as a consequence.

There is a need in the art to improve the properties of plant protein comprising food. The present patent application addresses this need.

### FIGURES

FIG. 1: Visual inspection of the fermented soy beverage (upper panel, A) and the fermented pea beverage (lower panel, B) processed without phytase enzyme (1) or with phytase enzyme at 6 FTUg/DW (2) or 20 FTU/g DW (3).

FIG. 2: Visual inspection of pea protein solution incubated with phytase enzyme: bottle left—no enzyme added during incubation; bottle right—with 20 FTU/g DW phytase added.

### SUMMARY

The invention provides:
a method for preparing a plant protein comprising food product with an improved property, said method comprising
incubating a solution comprising a plant protein with phytase (to obtain a phytase-treated solution) and
optionally processing the phytase-treated solution into a food product comprising a plant protein;
a method for producing a plant protein based cheese, comprising incubating a plant protein solution with phytase and allowing said phytase to coagulate proteins in said plant protein solution;
a food product obtainable by a method as described herein;
use of phytase for improving taste, texture and/or mouthfeel of a food product comprising a plant protein, preferably
   for increasing gel-like structure with increased water binding, creaminess, sweetness, gelation or smoothness of a food product comprising a plant protein, or
   for decreasing pea taste, syneresis, astringency or beany taste of a food product comprising a plant protein, or
   for decreasing or increasing viscosity of a food product comprising a plant protein, or
   for coagulating plant proteins, preferably pea proteins.

### DETAILED DESCRIPTION

At present, the texture and taste of plant protein based food is obtained by adding stabilizers and thickeners such as hydrocolloids to improve texture or flavors to mask off flavors.

Surprisingly, the use of stabilizers, thickeners or flavors to plant protein based food products can be reduced by using phytase in the production process of plant protein based products. Preferably, the use of stabilizers, thickeners or flavors can be completely omitted such that a clean label product can be offered.

As a result, this invention results in a larger acceptation of plant-based food products due to taste and texture improvement. Minimally processed plant based food products increase acceptance and trust of consumers on plant based foods alternatives to dairy products.

It is surprising that phytase can improve taste, texture and/or mouthfeel in plant protein based food.

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

In one aspect, the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein.

Differently phrased, the invention provides a method for obtaining an improved property in a food product comprising a plant protein or a method for reducing the amount of off flavor masking compounds in a food product comprising a plant protein or a method for reducing the amount of stabilizers and thickeners such as hydrocolloids in a food product comprising a plant protein or a method for improving acceptance of a food product comprising a plant protein or a method for improving taste, texture and/or mouthfeel in a food product comprising a plant protein, any such method comprising
incubating a solution comprising a plant protein with phytase (to obtain a phytase-treated solution) and
optionally processing the phytase-treated solution into a food product comprising a plant protein.

The phrases “food product comprising a plant protein” and “plant protein comprising food product” are used interchangeably herein and refer to a food product which comprises at least 10% (based on all proteins present in said food product) plant protein. Preferably, a food product comprising a plant protein comprises at least 20%, 30%, 40%, 50%, 60%, 70%, 80% or 90% (based on all proteins present in said food product) plant protein. Most preferably, a plant protein comprising food product comprises only or exclusively (i.e. 100% based on all protein present in said food product) plant protein and no animal protein at all.

The phrases “solution comprising a plant protein” and “a plant protein comprising solution” are used interchangeably herein and refer to a liquid composition which comprises a plant protein. Such a liquid composition is for example prepared by dissolving (or diluting depending on the state of matter of the starting material) a plant flour or plant protein concentrate or a plant protein isolate into a suitable liquid (for example water or a buffered solution). The concentration of plant protein in the plant protein comprising solution will for example depend on the desired food product (for example, a low protein or a high protein drink) but also on the starting material. The skilled person is able to prepare a solution comprising a plant protein with a suitable amount of plant protein. The concentration of plant protein in the plant protein solution is at least 0.5% (w/w), preferably at least 1% (w/w), more preferably at least 1.5% (w/w) or 2% (w/w) and most preferred at least 2.5% (w/w).

Preferably, a method of the invention starts from a plant flour, plant protein concentrate or plant protein isolate. In case a flour, concentrate or isolate is not available, the skilled person can also start from plant grains, peas, beans, nuts or seeds and process them in a drink according to well-known methods which typically involve steps like (i) cleaning, soaking and/or dehulling, (ii) grinding of the starting material to produce a slurry, powder or emulsion, (iii) heating to inactivate endogenous enzymes, (iv) filtration to remove solids, (v) adding of water and optionally sugar, (vi) pasteurization, (vii) homogenization and/or (viii) packaging, labelling and/or storage.

The preparation of a plant protein comprising solution may comprise mild (such as to avoid protein degradation) heating for a certain time and/or stirring for a certain time to ensure that the flour, protein concentrate or protein isolate is completely dissolved and a homogenous plant protein solution is obtained before the incubation with phytase takes place.

A method of the invention can thus comprise an additional step which comprises dissolving flour or powder to obtain a solution comprising a plant protein. Such a dissolving step typically comprises heating and/or stirring.

The “plant protein comprising solution” may be subjected to a heat treatment to reduce the amount of micro-organisms and to extend the shelf life of the final food product. Suitable heat treatments are pasteurization or ultra-high temperature (UHT) treatment.

The term “phytase” as used herein refers to an enzyme that is capable of breaking down phytic acid/phytate (inositol hexaphosphate), by hydrolyzing phytic acid/phytate so as to release at least one phosphate group from phytate.

Phytases are widespread in nature and have been found in bacteria, yeasts, fungi and plants and phytases from any of these may be used in the invention. The fungal enzyme phytase from Aspergillus niger has been commercialized for use in animal feed and may also be used in the present invention. The gene encoding the enzyme has been cloned and the phytase enzyme has been overexpressed in Aspergillus niger. This fungus is grown on industrial scale in large fermentors allowing for the production of the enzyme. The fungus secretes considerable amounts of phytase which can be separated from the biomass in a series of filtration and ultrafiltration steps. The resulting concentrated ultrafiltrate is subsequently formulated into a stable granulate or liquid which may be used in the present invention.

The step of “incubating a solution comprising a plant protein with phytase” is performed under conditions such that phytase is able to perform its enzymatic activity. The skilled person is aware that the conditions for incubation depend for example on the specific enzyme used, enzyme concentration, time, temperature, pH etc. In the present invention phytase is typically incorporated in an amount of at least 0.1 FTU per gram flour, isolate or concentrate; one FTU being the activity of phytase that generates 1 μmol of inorganic phosphorus per minute from an excess of sodium phytate at pH 5.5 and 37° C. A suitable methodology for determining phytase activity is described in M. Wyss et al.; Biophysical Characterisation of fungal phytases (myo-Inositol hexakisphosphate phosphohydroiase): Molecular size, glycosylation pattern, and engineering of proteolytic resistance. Appl. Envir. Micr., 65 (2), 359-366, 1999. Preferably, phytase is incorporated in an amount of at least 0.5, 1.0, 1.5 or 2.0 FTU per gram flour, isolate or concentrate.

The phytase is added to a solution comprising a plant protein and subsequently incubated such as to obtain a phytase-treated solution. I.e. the invention provides a method for preparing a plant protein comprising food product with an improved property, said method comprising
incubating a solution comprising a plant protein with phytase to obtain a phytase-treated solution and
optionally processing the phytase-treated solution into a food product comprising a plant protein.

The step of “incubating a solution comprising a plant protein with phytase” results in a phytase treated solution. The phytase treated solution can be subjected to an enzyme (phytase) inactivation step, for example by heating the phytase treated solution to 90° C. and keeping at 90° C. for 15 minutes. The inactivation step can also be combined with a heat treatment step to reduce the amount of micro-organisms.

The phytase treated solution can be the final food product. This is typically the case when the food product is a drink or plant milk or beverage.

A method as described above may comprise an optional step of processing the phytase-treated solution into a food product comprising a plant protein. Examples of a step wherein the phytase-treated solution is processed into a food product comprising a plant protein are:
fermenting the phytase-treated solution by adding lactic acid bacteria and allowing the lactic acid bacteria to ferment the phytase-treated solution such as to obtain a fermented food product,
processing the phytase-treated solution into a meat alternative,
homogenizing (for example used in the preparation of a stirred yogurt process or as used in the preparation of a drink),
emulsifying,
performing a solid-liquid separation, or
processing the phytase-treated solution into ice cream.

A method of the invention may also comprise a step of adding an ingredient, such as
adding of a micronutrient such as a vitamin ora mineral (for example calcium),
adding of (reduced amounts oh a stabilizer or a hydrocolloid,
adding of oil,
adding of (reduced amounts oh flavour components,
adding of a fruit preparation.

A method of the invention can further comprise a step of packaging the food product.

In one its embodiments, the invention provides a method as described herein wherein said method excludes the addition of a hydrocolloid or a stabilizer such that a clean label product can be prepared.

A method of the invention can be used to prepare different kind of food products, for example the food product is
a drink (i.e. a drink comprising a plant protein),
a fermented food product (i.e. a fermented food product comprising a plant protein),
ice cream (i.e. an ice cream comprising a plant protein), or
cheese (i.e. a dairy cheese alternative/a non-dairy cheese/a plant protein based cheese)

Examples of a drink are a milk, preferably a dairy milk alternative, a fortified drink, a drink with a neutral or acidic pH or a sport drink. Yet another example of a drink is a dairy shake alternative. Preferably, the drink is a plant drink. The fortified drink is for example a protein fortified drink or a vitamin fortified drink.

Examples of a fermented food product are a yogurt, preferably a dairy yogurt alternative (plant protein based yogurt) or cheese.

As used in the present specification, the term “fermented food product” refers to a product that has been fermented with lactic acid bacteria such as Streptococcus thermophilus and optionally Lactobacillus delbruekii subsp. bulgaricus, but also, optionally, other microorganisms such as Lactobacillus delbruekii subsp. lactis, Bifidobacterium animalis subsp. lactis, Lactococcus lactis, Lactobacillus acidophilus and Lactobacillus casei, or any microorganism derived therefrom. The lactic acid strains other than Streptococcus thermophilus and Lactobacillus delbruekii subsp. bulgaricus, are typically intended to give the finished product various properties, such as the property of promoting the equilibrium of the flora. The fermentation process increases the shelf-life of the product while enhancing and improving the digestibility of the food product. Many different types of fermented milk products can be found in the world today.

As used herein, the term “yogurt” is a fermented plant milk product produced by fermentation of milk by lactic acid bacteria, also known as “yogurt cultures”. The fermentation of the (added) sugars in the plant milk produces lactic acid which acts on the plant milk protein to give the yogurt its texture. The plant milk is for example obtained by lactic acid fermentation by means of specific thermophilic lactic acid bacteria only (i.e. Lactobacillus delbruekii subsp. bulgaricus and Streptococcus thermophilus) which are cultured simultaneously and are found to be living in the final product in an amount of at least 10 million CFU (colony-forming unit) per gram of the yogurt. Preferably, the plant yogurt is not heat-treated after fermentation. Plant yogurts may optionally contain other ingredients such as sugar or sweetening agents, one or more flavouring(s), cereals or nutritional substances, especially vitamins, minerals and fibers.

Yogurt encompasses set yogurt, stirred yogurt, drinking yogurt, Petit Suisse, heat treated yogurt and yogurt-like products. Preferably, the yogurt is a stirred yogurt or a drinking yogurt. More preferably, the yogurt is a stirred yogurt.

The term “starter culture composition” or “composition” (also referred to as “starter” or “starter culture”) as used herein in the experimental part refers to a composition comprising one or more lactic acid bacteria, which are responsible for the acidification of the plant milk base. Starter cultures compositions may be fresh (liquid), frozen or freeze-dried. Freeze dried cultures need to be regenerated before use. The skilled person is capable of determining the amount of starter culture which needs to be added. For the production of a fermented plant product, the starter cultures composition is added in an amount of at least 0.01% by weight of the total amount of plant milk base.

As used herein, the term “lactic acid bacteria” (LAB) or “lactic bacteria” refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of lactose by these bacteria causes the formation of lactic acid, reduces the pH and leads to the formation of a protein coagulum. These bacteria are thus responsible for the acidification of plant milk and for the texture of the fermented plant milk product. As used herein, the term “lactic acid bacteria” or “lactic bacteria” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus thermophilus, Lactobacillus lactis, Bifidobacterium animalis, Lactococcus lactis, Lactobacillus casei, Lactobacillus plantarum, Lactobacillus helveticus, Lactobacillus acidophilus and Bifidobacterium breve. Preferably, the used culture composition comprises Lactobacillus delbruekii subsp. bulgaricus and Streptococcus thermophilus.

The property which is preferably improved in a food product comprising a plant protein is texture, taste or mouthfeel.

The term “mouthfeel” refers to the physical sensation in the mouth caused by food or drink and is distinct from taste. Mouthfeel is a fundamental sensory attribute which, along with taste and smell, determine the overall flavour of a food product.

The term “taste” refers to the perception produced or stimulated when a substance in the mouth reacts chemically with taste receptor cells located on taste buds in the oral cavity, mostly on the tongue.

The term “texture” refers to visual or mechanical assessment of a food product.

Examples of a property are gel-like structure with increased water binding, viscosity, creaminess, sweetness, grass taste, syneresis, smoothness, astringency or beany taste.

Examples of a texture property are gel-like structure with increased water binding, viscosity, smoothness or syneresis.

Example of a mouthfeel property are creaminess or smoothness.

Examples of a taste property are sweetness, grass taste, astringency or beany taste.

It is also possible to have combined properties, for example texture and mouthfeel. An example of texture and mouthfeel (i.e. combined properties) is smoothness.

Whether or not a property is improved can be easily determined by comparing a method of the invention with a similar method in which phytase is not used but all other conditions are identical. An improvement in one or more of the properties related to texture, taste or mouthfeel may be measured absolutely for instance in the case of Brookfield (Pa*s units) or shear stress (Pa units) or more relatively by a taste panel for instance for all the sensory aspects of the food product. I.e. the invention provides a method as described herein, wherein the food product has an improved property as compared to a food product comprising a plant protein that has not been prepared in the presence of phytase but which food product has otherwise been prepared identically. The improvement of a property is at least 2%, 3%, 5%, 10%, for instance at least 20%, 25%, 30%, 35%, 40%, 45%, 50%, 55%, 60%, 65%, 70%, 75%, 80%, 85% or at least 90% or 95%. The improvement of a property of a plant protein comprising food product that has been prepared with phytase is compared to a plant protein comprising food product that has not been incubated with phytase under the same conditions.

Preferably, the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein,
wherein said improved property is increased gel-like structure with increased water binding, decreased viscosity, increased creaminess, increased sweetness, decreased pea flavour, decreased syneresis (also referred to as increased gelation), increased smoothness, decreased astringency or decreased beany taste.

The term gel-like structure with increased water binding as used herein refers to a solid-like structure containing dispersed particles in a dilute system (in general more than 80% liquid) which exhibits no flow in the steady state.

The term “viscosity” as used herein refers to the state of being thick, sticky, and semi-fluid in consistency, due to internal friction. Determination of the viscosity of a fermented milk product is well known to the skilled person. A well accepted method is the use of a Brookfield viscometer.

The term “creaminess” as used herein refers to degree in which the product gives a full and creamy mouthfeel (the product doesn't feel rough, it's not dry and it has a velvety coating). Can for example be determined by putting a spoon of food product (for example yoghurt) in the mouth. Manipulate the product by making chewing/rolling movements against the palate.
The term “sweetness” as used herein refers to the basic taste most commonly perceived when eating foods rich in sugar.
The term “pea flavour” as used herein refers to the typical earthy or grass taste and smell of pea products.

The terms “decreased syneresis” and “improved gelation” are used interchangeably herein and refer to the decreased expulsion of a liquid from a gel.

The term “smoothness” as used herein refers to the degree in which the product is smooth without grittiness, grains and lumps. This can for example be judged by lifting a spoon and assess the back of the spoon.

The term “astringency” as used herein refers to the dry, puckering mouthfeel similar to that caused by tannins that are for example found in many fruits or by proteins, especially in an acidic environment. Can for example be determined by the degree in which an astringent afterfeel is present in the mouth after the product is swallowed.

The term “beany taste” as used herein refers to an off-flavour suggestive of that of beans.
As used herein, the term “plant protein” refers to any protein from plant origin. Preferably, the plant protein is a protein from grains, pseudocereals, legumes, nuts, seeds or other sources such as coconut, potato, canola or tiger nut.

Examples of suitable grains are barley, fonio, maize, millet, oat, rye, sorghum, teff, triticale, spelt, rice or wheat.

Examples of suitable pseudograins are amaranth, buckwheat or quinoa.

Examples of suitable legumes are lupin, pea, chickpea, beans (preferably faba beans), peanut or soy.

Examples of suitable nuts are almond, brazil, cashew, hazelnut, macadamia, pecan, pistachio or walnut.

Examples of suitable seeds are chia seed, flax seed, hemp seed, pumpkin seed, sesame seed or sunflower seed.

The plant protein may comprise a blend of proteins which is prepared by mixing two or more plant protein types, for example by mixing almond and coconut proteins or by mixing almond and cashew proteins. I.e. in one of the embodiments, the solution comprising a plant protein comprises plant proteins from at least 2 different types of plants.

As mentioned above, a method of the invention preferably starts from a plant flour, plant protein concentrate or plant protein isolate. The preferred starting material depends, amongst others, on the amount of proteins present in a particular plant flour, plant protein concentrate or plant protein isolate. For example, rice or oat flour typically has a low protein concentration and hence it is preferred to start from rice protein concentrate, rice protein isolate, oat protein concentrate or oat protein isolate.

The inventors of the present invention show multiple surprising improved properties (detailed in experimental part herein) when phytase is used on pea protein, almond protein, oat protein or soy protein.

In one of its embodiments, the invention provides surprising improved properties on pea protein and hence the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein,

wherein said plant protein is pea protein and
(i) wherein said food product is a drink and wherein said improved property is increased creaminess, increased sweetness or decreased pea flavour, or
(ii) wherein said food product is a fermented food product and wherein said improved property is decreased syneresis, decreased viscosity, increased smoothness, decreased pea taste or decreased astringency.

In yet another embodiment, the invention provides surprising improved properties on almond protein and hence the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein,

wherein said plant protein is almond protein and
(i) wherein said food product is a drink and wherein said improved property is decreased astringency or improved mouthfeel (less sand like), or
(ii) wherein said food product is a fermented food product and wherein said improved property is decreased viscosity or improved taste.
In one of its aspects, the invention provides surprising results on pea protein and hence the invention provides

In yet another embodiment the invention provides surprising improved properties on soy protein and hence the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein,

wherein said plant protein is soy protein and
(i) wherein said food product is a drink and wherein said improved property is increased smoothness or decreased astringency, or
(ii) wherein said food product is a fermented food product and wherein said improved property is decreased syneresis, decreased viscosity or decreased beany taste.

In yet a further embodiment the invention provides surprising improved properties on oat protein and hence the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant protein,
   wherein said plant protein is oat protein and wherein said food product is a fermented food product and wherein said improved property is decreased viscosity.

It is shown within the experimental part that phytase is capable of coagulating plant proteins like a coagulant (for example chymosin) is capable of coagulating milk proteins. The invention therefore also provides a method for producing a plant protein based cheese, comprising incubating a plant protein solution with phytase and allowing said phytase to coagulate proteins in said plant protein solution. Alternatively phrased, the invention provides a method for preparing a plant protein comprising food product with an improved property, said method comprising
incubating a solution comprising a plant protein with phytase (to obtain a phytase-treated solution) and
optionally processing the phytase-treated solution into a food product comprising a plant io protein,
wherein said food product is a plant protein based cheese and wherein said improved property is increased cheese texture.

Some of the wordings and phrasings (for example “plant protein solution” and “phytase”) have already been explained above and are applicable to this part as well.

The terms “dairy cheese alternative” and “non-dairy cheese” or “dairy-free cheese” or “plant protein based cheese” are used interchangeably herein and refer to a cheese which does not comprise any dairy protein.

The phytase is allowed sufficient time to coagulate the plant protein which is present in the plant protein solution. The phytase is added to the plant protein solution at a dose of at least 0.5 FTU/g DW, but preferably at least 1 or 2 or 5 or 10 or 15 or 20 FTU/g DW is used. The solution is incubated with the enzyme at a temperature of at least 4° C. for at least 10 hours or at 40° C. for 1 hour, before heating to 95° C. to inactivate the enzyme.

A method for producing a plant-based cheese typically comprises a step wherein the resulting (by the action of phytase) solids and liquids are separated, i.e. a method for producing a dairy cheese alternative, further comprises a solid-liquid separation step.

Techniques to separate solids from liquid are well-known to the skilled person and include filtration techniques or the use of cheese cloth or the use of a centrifuge.

Suitable examples of plant proteins have been described above. In a preferred embodiment, the plant protein is pea protein.

The preparation of a fermented food product (for example yogurt or cheese) typically comprise a step of adding lactic acid bacteria to ferment the phytase-treated solution such as to obtain a fermented food product. Suitable lactic acid bacteria are described above. In case the food product is a fermented food and hence a fermentation step is included in a method of the invention, the phytase is added before or during fermentation. For example, the phytase can be added to the solution comprising a plant protein and is subsequently incubated such as to obtain a phytase-treated solution and the lactic acid bacteria are added to said phytase-treated solution and are subsequently allowed to acidify the phytase-treated solution to obtained a fermented food product. Alternatively, the lactic acid bacteria and the phytase are added to the solution comprising a plant protein and is subsequently incubated such as to obtain a fermented food product. In case a plant protein solution does comprise sufficient levels of carbon source, a sugar can be added for the cultures to grow. Suitable examples of a sugar are sucrose or glucose or lactose.

Optional steps which can be part of the method of the invention have been described above. The use of enzymes in the preparation of a food product comprising a plant protein is known. As a non-limiting example, reference is made to the production of oat milk which is similar to that of most other plant drinks. The process typically start with milling of oat grains to break the outer hull, then the grains are stirred in warm water and ground into a slurry. The slurry is treated with enzymes and heated to create a thick liquid oat base which can be further processed into the final oat drink or fermented oat product. Enzymatic hydrolysis of starch by alpha- and/or beta-amylase, producing maltodextrins (liquefaction) and subsequent hydrolysis of the dextrins to glucose by amyloglucosidase (gluco-amylase) (saccharification) or hydrolysis by alpha-amylase of dextrins to maltose (saccharification as well) are well known. Thus in one of its aspects, a method of the method further comprises incubating the solution comprising a plant protein with at least one starch degrading enzyme. Preferably, said solution is first incubated with at least one starch degrading enzyme and subsequently with phytase. Examples of a suitable starch degrading enzyme are alpha-amylase, beta-amylase or gluco-amylase. Preferably, a method of the invention further comprises incubating the solution comprising a plant protein with at least two (preferably alpha-amylase and glucoamylase) or three starch degrading enzymes. Alpha-amylase, beta-amylase or gluco-amylase are commercially available and can thus be used by the skilled person in a method of the invention. Preferably, a method of the invention further comprises incubating the solution comprising a plant protein with at least two (preferably alpha-amylase and glucoamylase) or three starch degrading enzymes, such that liquefaction and saccharification can be performed in one step.

A method of the invention can further comprise the use of other enzymes, for example a peptidyl arginine deiminase (PAD).

The term protein arginine deiminase and peptidyl arginine deiminase (PAD) are used interchangeably herein. Protein or peptidyl arginine deiminases belong to a family of enzymes (EC 3.5.3.15) which convert peptide or protein bound arginine into peptide or protein bound citrulline. This process is called deamination or citrullination. In the reaction from arginine to citrulline, one of the terminal nitrogen atoms of the arginine side chain is replaced by an oxygen. The reaction uses one water molecule and yields ammonia as a side product (http://en.wikipedia.org/wiki/Citrullination). Whereas arginine is positively charged at a neutral pH, citrulline is uncharged. Surprisingly, it was found that a protein wherein at least part of the arginine has been converted into citrulline, and thereby resulting in protein with less charge, exhibited modified sweetness, liquorice, astringency, powdery/chalk, fulness, thickness and/or digestibility.

Peptidyl arginine deiminase (PAD) may be derived from any suitable origin, for instance from mammalian or microbial origin. PAD's used in the present invention are advantageously derived from a microbial source, i.e. the PAD used in a process of the invention is a microbial PAD. For instance, PAD's may be derived from fungal origin such as from Fusarium sp. such as Fusarium graminearum, Chaetomium globosum, Phaesphaeria nodorum or from bacterial origin such as from the bacteria Streptomyces, eg Streptomyces scabies, Streptomyces clavuligeres. The wording “derived” or “derivable” from with respect to the origin of a polypeptide as disclosed herein, means io that when carrying out a BLAST search with a polypeptide as disclosed herein, the polypeptide may be derivable from a natural source, such as a microbial cell, of which an endogenous polypeptide shows the highest percentage homology or identity with the polypeptide as disclosed herein.

Peptidyl arginine deiminases are for instance known from WO2008/000714. WO2008/000714 discloses a process for enzymatically treating a protein with a protein arginine deiminase, wherein at least 30% of the arginine is transformed into citrulline.

PAD is typically added in an amount of 0.1% enzyme protein to protein substrate and incubated at a suitable temperature and pH, for instance incubating pea protein with a peptidyl arginine deiminase at a pH of between 4 and 9, such as a pH of between 5 and 8.5, such as a pH of between 5.5 and 8, such as a pH between 6 and 7, or a pH of between 6.2 and 6.8, for instance at a pH of about 6.5. A suitable temperature at which protein is incubated with PAD may be between 20 and 60 degrees Celsius, such as a between 30 and 50, or between 35 and 45 degrees Celsius. Peptidylarginine deiminase activity was determined by measuring the formation of citrulline residues in α-N-Benzoyl-L-arginine-ethyl ester (BAEE). The incubation mixture contained 100 mM tris-HCl buffer (pH 7.5), 5 mM CaCl₂, 10 mM DTT, 10 mM BAEE in a final volume of 700 μl. Incubation was performed at 55° C. for 30 min, and the reaction was stopped by adding 100 μl 8 N HClO₄. Citrulline was determined by colorimetry according the method of Guthöhrlein and Knappe, (1968) Anal. Biochem. 26, 188. One unit of peptidyl arginine deiminase is expressed as 1 μmol of citrulline formed/min/mg of protein.

In yet another aspect, the invention provides a food product obtainable by a method of the invention, i.e. by a method for preparing a plant protein comprising food product with an improved property, said method comprising
incubating a solution comprising a plant protein with phytase (to obtain a phytase-treated solution) and
optionally processing the phytase-treated solution into a food product comprising a plant protein.

Examples of food products are described above and apply to this part of the invention as well. A food product of the invention can be distinguished from prior art food products in view of the (i) phytate content or (ii) reduced levels of thickeners or stabilizers or (iii) absence of thickeners or stabilizers

In a further aspect the invention provides use of phytase for improving a (taste or texture/structure) property of a food product comprising a plant protein.

In one of its aspects, the invention provides use of phytase for increasing gel-like structure with increased water binding, creaminess, sweetness, gelation or smoothness of a food product comprising a plant protein.
In another aspect, the invention provides use of phytase for decreasing pea taste, syneresis, astringency or beany taste of a food product comprising a plant protein.

In a further aspect, the invention provides use of phytase for decreasing or increasing viscosity of a food product comprising a plant protein. The effect of phytase on viscosity depends on the source (plant) of the protein but also on specific conditions used. The use of phytase for the production of a fermented food product typically result in a decreased viscosity. An increased viscosity is useful because the amount of stabilizer can be reduced.

It is clear from the examples in this patent application, some more detailed aspects can be defined:
Use of phytase for increasing creaminess or sweetness in a pea protein drink.
Use of phytase for decreasing pea taste in a pea protein drink.
Use of phytase for increasing smoothness in a fermented pea protein food product.
Use of phytase for decreasing syneresis, viscosity, pea taste or astringency in a fermented pea protein food product.
Use of phytase for improving mouthfeel of an almond drink.
Use of phytase for decreasing astringency in an almond drink.
Use of phytase for decreasing viscosity in a fermented almond protein food product.
Use of phytase for improving taste in a fermented almond protein food product.
Use of phytase for increasing smoothness in a soy protein drink.
Use of phytase for decreasing astringency in a soy protein drink.
Use of phytase for decreasing syneresis, viscosity or beany taste in a fermented soy protein food product.
Use of phytase for decreasing viscosity in a fermented oat protein food product.
Use of phytase for coagulating plant proteins, preferably pea proteins.

The definitions and explanations which are given above for the methods claims apply to the use claims as well.

The invention will be explained in more detail in the following example, which are not limiting the invention.

### EXPERIMENTAL PART

The Production of Plant-Derived Beverages (Fermented and Non-Fermented) Processed with Phytase

Dairy alternatives (beverage and yoghurt) were produced from pea, soy and almond protein ingredients. The pea isolate, Pisane M9 was obtained from Cosucra, the soy isolate, Profam 974 was obtained from Archer Daniels company and the almond flour was purchased from notenshop.nl. The three different raw materials (pea isolate, soy isolate and almond flour) were processed into beverage by dissolving the powders in tap water with 2% w/w sucrose added, high speed mixing for 10 min at 40° C. followed by a heat step of 30 min at 85° C.

For the oat beverages, 10% and 15% flour solutions (oat flour from Alifarma, Spain) were io made in water followed by the addition of a starch hydrolysing enzyme, amyloglucosidase in order to release glucose (between 1-3% glucose generated in the beverage). No additional sucrose was added to the oat bases before the phytase incubation.

For the soy and pea beverage, a 4 w % protein solution in tap water was processed, assuming isolates with 94 and 86% protein, respectively, as indicated in specification sheets. For the almond beverage, a 10% dry weight solution was selected as suitable for processing, resulting in about 2.1 w % protein in the beverage. Processing was performed in the Thermomixer (TM5).

Phytase (DSM) was added in doses of 6 and 20 FTU/gram isolate/flour in the beverage, or 30 FTU/g flour in the oat beverages. The beverage was left for incubation at 4° C. for 17 hours. For phytase inactivation, the beverage was heated up to 90° C. and held at 90° C. for 15 min after which it was cooled on ice.

A portion of these beverage alternatives obtained from the process described above was further analysed for enzyme conversion, texture and taste. The rest of the product was further processed into a yoghurt like plant alternative.

The 12 different plant-based beverages (4 raw materials with 2 to 3 different phytase incubations from previous step) were acidified at 42° C. with culture DelvoFresh YS-141 (this culture comprises Streptococcus thermophilus and Lactobacillus bulgaricus). A 2 U bag YS-141 was dissolved in saline and from there added to the beverage, dosing 1.8 U/1000 kg. For each beverage one CINAC (˜80 ml) and three cups (˜125 ml) were used. CINAC acidifications were performed in a water bath set at 42° C., acidification in cups was performed in a stove set at 43° C. After reaching pH 4.6 or lower, fermentation was stopped, and samples were kept at 4° C.

### EXAMPLE 1

Phytic Acid Conversion with Phytase in the Process of Plant Beverage Production

Phytate analysis of samples was performed using 31P-NMR.

Of each beverage, a sample of 2 g was mixed with 12.5 ml 0.5 M HCl, mixed thoroughly and put in a sonication bath (Branson 5510) for 30 minutes. Samples of 2×2 ml of each suspension were transferred into Eppendorf centrifuge tubes and centrifuged during 15 min at 17949 relative centrifugal force (rcf). 2000 μl of the supernatants and 500 μl of an Internal Standard solution were mixed (standard solution contains: +/−25 mg phosphonoacetic acid, 2.25 g EDTA, 6.5 ml D2O and 2.5 ml 6 N KOH in a 25.0 ml volumetric flask, replete with demineralized water) and pH was set to pH7.5. After lyophilization the residues were resuspended in 1 ml of D2O. The pH of the solutions was adjusted to pH 12.5 prior to the NMR measurements.

TABLE 1
phytic acid content in the plant-based
beverages incubated with phytase enzyme

| | Phytase enzyme, | IP6 *(phytate), | Phosphate, |
|---|---|---|---|
| Sample name | FTU/g DW | g/kg DW | g/kg DW |
| Pea beverage | 0 | 14.6 | 0.7 |
| | 6 | 0 | 11.6 |
| | 20 | 0 | 12 |
| Soy beverage | 0 | 5.9 | 0.4 |
| | 6 | 2.9 | 2.6 |
| | 20 | 2.6 | 3.1 |
| Almond beverage | 0 | 2.4 | 0.1 |
| | 6 | 0 | 2 |
| | 20 | 0 | 2.2 |
| Oat beverage, | 0 | 6 | 1 |
| (15% flour) | 30 | 0 | 6 |

*myoinositol, 1, 2, 3, 4, 5, 6 hexakis-dihydrogen phosphate or inositol hexakishosphate

Pea isolate has about 15 g phytate/kg and soy isolate about 6 g phytate/kg. For almond, phytate content is about 2.5 g phytate /kg flour and about 6 g phytate/kg oat flour. For the pea and almond beverages total conversion of phytate is achieved already using a 6 FTU/g DW phytase dose. For the soy samples only about half of phytate is converted in the conditions tested. For the oat beverage, complete conversion of phytic acid is achieved in the product with 30 FTU/g DW enzyme.

### EXAMPLE 2

Sensory Assessment of the Non-Fermented Plant Beverages

Plant based beverage and yoghurts were tasted by an internal sensory panel (N>4), and large effects were noted by all panellist regarding taste and texture (Table 2).

TABLE 2
sensory assessment of nonfermented plant based beverages

| | | |
|---|---|---|
| Beverage | | |
| with phytase | Beverage taste | Beverage mouthfeel |

Pea

| | | |
|---|---|---|
| 0 | Pea (bean and grass) | Chalk |
| | taste, slightly bitter | |
| 20 FTU/g DW phytase | Sweeter, less pea taste | Creamy, thick |
| | | mouthfeel |

Soy

| | | |
|---|---|---|
| 0 | Sweet | Sweet |
| 6 FTU/g DW phytase | Less astringent | Smoother and less |
| | | mouthfeel |
| 20 FTU/g DW phytase | Less astringent | Smoother and less |
| | | mouthfeel |

Almond

| | | |
|---|---|---|
| 0 | Sweet | Grainy/sand like |
| 6 FTU/g DW phytase | Sweet | Grain/less sand like |
| 20 FTU/g DW phytase | Less astringent | Less sand like |

### EXAMPLE 3

Viscosity Measurements in Fermented Plant Beverages

Viscosity of samples was measured with the Brookfield RVDV-II+Pro Viscometer (with Helipath). For the soy and pea yoghurt samples, a time sensitivity test was performed, in which at 31 points in time (3 sec interval) viscosity was measured with spindle T-C at speed 30 rpm. Before starting, the spindle was lowered until just on top of the surface of the yoghurt. For each yoghurt 2 cups were used which were kept cold until analysis.

Viscosity of almond yoghurt samples was measured using spindle RV-3 (measurement without Helipath). At 21 points in time (3 sec interval) viscosity was measured at speed 30 rpm. Before starting, the spindle was lowered into the samples up until the spindle shaft indentation. For each yoghurt 2 cups were used which were kept cold until analysis, for each beverage the sample was poured in one 125 ml cup and measured.

The data can be plotted (viscosity vs measurement time); Final viscosity is defined as the average viscosity over the final 30 seconds of the measurement (10 datapoints). The viscosity reduction with the enzyme added was calculated from the ratio of viscosity in the fermented beverage and the fermented beverage with enzyme preincubation.

TABLE 3
viscosity reduction of the fermented plant beverages

| | | Fold viscosity |
|---|---|---|
| | Phytase enzyme, | reduction |
| Sample name | FTU/g DW | by phytase |
| Fermented pea beverage | 0 | — |
| | 6 | 2.3 |
| | 20 | 2.3 |
| Fermented soy beverage | 0 | — |
| | 6 | 1.2 |
| | 20 | 1.2 |
| Fermented almond beverage | 0 | — |
| | 6 | 1.2 |
| | 20 | 1.2 |
| Fermented oat beverage | 0 | — |
| (15% oat flour) | 30 | 2.02 |

In all fermented beverages a reduction of viscosity of at least 15% was obtained with the phytase enzyme preincubation.

### EXAMPLE 4

Sensory Assessment of the Fermented Plant Beverages

Plant based beverage and yoghurts were tasted by an internal sensory panel (N>4), and large effects were noted by all panellist regarding taste and texture (Table 4).

TABLE 4
sensory assessment of fermented plant based beverages

| | |
|---|---|
| Fermented beverage | |
| with phytase | Fermented beverage sensory |

Pea

| | |
|---|---|
| 0 | Firm, syneresis |
| 6 FTU/g DW phytase | More smooth, fresh taste/more acidic, |
| | less pea taste, less astringent |
| 20 FTU/g DW phytase | More smooth, fresh taste/more acidic, |
| | less pea taste, less astringent |

Soy

| | |
|---|---|
| 0 | Firm, syneresis |
| 6 FTU/g DW phytase | More acidic, less bean taste, less |
| | syneresis, neutral and mild taste |
| 20 FTU/g DW phytase | More acidic, less bean taste, less |
| | syneresis, neutral and mild taste |

Almond

| | |
|---|---|
| 0 | Rancid, acidic |
| 6 FTU/g DW phytase | Less rancid off flavour, less acidic taste |
| 20 FTU/g DW phytase | Less rancid/off flavour, less acidic taste |

### EXAMPLE 5

Syneresis Reduction of the Fermented Plant Beverages.

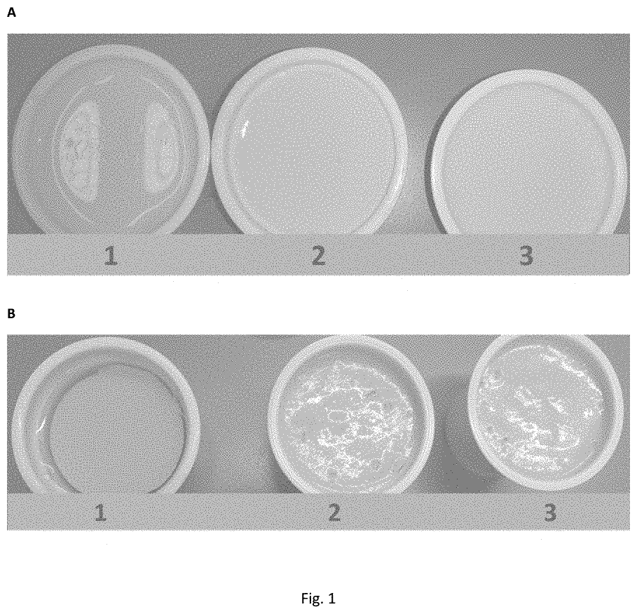

The fermented beverages described above were inspected for the amount of syneresis (the expulsed liquid from the gel structure settled on top of the fermented beverage). A clear syneresis reduction was observed in the fermented plant beverages which were preincubated with the phytase enzyme. Results are shown in FIG. 1.

Significant syneresis was observed in the plant fermented beverages (above 10% of the total beverage volume). More than 3 fold reduction in the syneresis was measured in the fermented pea and soy beverages, measured as the ratio of the liquid volume decanted from the surface of the o fermented beverage without and with enzyme incubation.

### EXAMPLE 6

Pea Beverage Coagulation with Phytase

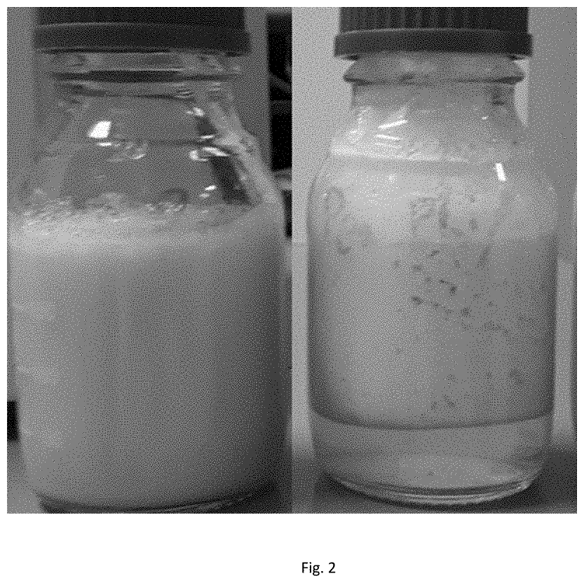

A 4% pea protein solution at pH 6.7 was made as described above. This solution was incubated with 20 FTU/g DW of phytase enzyme for 2 hours at 40° C. The incubation was then heated up at 90° C. for 15 min to inactivate the enzyme. A visual inspection of this material with and without enzyme incubation is shown in FIG. 2.

After enzyme incubation and heating, the pea solution increased in viscosity and gelled and significant syneresis was observed. A clear separation between the solid-liquid layers is obtained after the enzyme treatment. The initial pH did not change after incubation with or without the enzyme added. The pea solution incubated without enzyme addition did not gel.

1. A method for preparing a plant protein comprising food product with an improved property, said method comprising
incubating a solution comprising a plant protein with phytase, and optionally processing the phytase-treated solution into a food product comprising a plant protein.

2. The method according to claim 1, wherein said food product is a drink.

3. The method according to claim 2, wherein said drink is a dairy milk alternative, a fortified drink, a drink with a neutral or acidic pH or a medicinal or sport drink.

4. The method according to claim 1, wherein said food product is a fermented food product.

5. The method according to claim 1, wherein said improved property is increased gel-like structure with increased water binding, decreased viscosity, increased creaminess, increased sweetness, decreased pea flavour, decreased syneresis, increased smoothness, decreased astringency or decreased beany taste.

6. The method according to claim 1, wherein said plant protein is pea protein and
(i) wherein said food product is a drink and wherein said improved property is increased creaminess, increased sweetness or decreased pea flavour, or
(ii) wherein said food product is a fermented food product and wherein said improved property is decreased syneresis, decreased viscosity or increased smoothness or decreased pea taste, or decreased astringency.

7. The method according to claim 1, wherein said plant protein is almond protein and
(i) wherein said food product is a drink and wherein said improved property is decreased astringency or improved mouthfeel (less sand like), or
(ii) wherein said food product is a fermented food product and wherein said improved property is decreased viscosity or improved taste.

8. The method according to claim 1, wherein said plant protein is soy protein and
(i) wherein said food product is a drink and wherein said improved property is increased smoothness or decreased astringency, or
(ii) wherein said food product is a fermented food product and wherein said improved property is decreased syneresis, decreased viscosity or decreased beany taste.

9. The method according to claim 1, wherein said plant protein is oat protein and wherein said food product is a fermented food product and wherein said improved property is decreased viscosity.

10. A method for producing a plant protein based cheese, comprising incubating a plant protein solution with phytase and allowing said phytase to coagulate proteins in said plant protein solution.

11. The method according to claim 1, wherein said food product is a plant protein based cheese and wherein said improved property is increased (cheese) texture.

12. The method according to claim 10, further comprising solid-liquid separation.

13. The method according to claim 10, wherein said plant protein is pea protein.

14. The method according to claim 1, wherein said food product is a fermented food product and wherein said phytase is added before or during fermentation.

15. The method according to claim 1, further comprising dissolving flour or powder to obtain a solution comprising a plant protein.

16. The method according to claim 1, further comprising incubating the solution comprising a plant protein with at least one starch degrading enzyme.

17. The method according to claim 16, wherein said solution is first incubated with at least one starch degrading enzyme and subsequently with phytase.

18. The food product obtainable by a method according to claim 1.

19. A product comprising of phytase for improving taste, texture and/or mouthfeel of a food product comprising a plant protein, optionally
for increasing gel-like structure with increased water binding, creaminess, sweetness, gelation or smoothness of a food product comprising a plant protein, or
for decreasing pea taste, syneresis, astringency or beany taste of a food product comprising a plant protein, or
for decreasing or increasing viscosity of a food product comprising a plant protein, or
for coagulating plant proteins, optionally pea proteins.
